(19) United States
(12) Patent Application Publication
(10) Pub. No.: US 2003/0194050 A1
(43) Pub. Date: Oct. 16, 2003

(54) Multi modality X-ray and nuclear medicine mammography imaging system and method

(75) Inventors: Jeffrey Wayne Eberhard, Albany, NY (US); Abdalmajeid Musa Alyassin, Niskayuna, NY (US); Bernhard Erich Hermann Claus, Niskayuna, NY (US)

Correspondence Address: FOLEY AND LARDNER, SUITE 500, 3000 K STREET NW, WASHINGTON, DC, 20007, US

(73) Assignee: GENERAL ELECTRIC COMPANY, Schenectady, NY

(21) Appl. No.: 10/063,354

(22) Filed: Apr. 15, 2002

(51) Int. Cl. A61B 6/04

(52) U.S. Cl. 378/37

(57) ABSTRACT

A multi modality imaging system contains an X-ray imaging subsystem and a nuclear medicine imaging subsystem. The X-ray imaging subsystem may be a tomosynthesis subsystem. The system may be used for mammography imaging, such that the X-ray imaging subsystem and the nuclear medicine imaging subsystem are adapted to image a breast compressed by a breast compression paddle.

### BACKGROUND OF THE INVENTION

The present invention relates generally to an imaging system, and more particularly to an multi modality X-ray and nuclear medicine mammography imaging system.

Various multi modality imaging sensors are currently being developed. For example, an article by Mark B. Williams, et al. “Multimodality Imaging of Small Animals,” published on the internet at http://ej.rsna.org/ej3/0107-99.fin/dual99.htm describes an experimental, multi modality system for imaging small animals, such as mice and rats. This system combines a conventional, two dimensional X-ray imaging system with a conventional two dimensional nuclear medicine imaging system. Another article by Mark B. Williams, et. al., “Integrated CT-SPECT System For Small Animal Imaging” published on the internet at http://imaging.med.virginia.edu/mbwlab/ct_spect_ms.pdf describes an experimental multi modality X-ray computed tomography (CT) and nuclear medicine single photon emission computed tomography (SPECT) system for imaging small animals. However, these systems are not adapted to image human patients. The two dimensional images do not yield the optimum amount of information, while the tomography systems which can generate three dimensional (“3D”) images are complex because they require 360 degree angular scanning of the animal. Furthermore, the CT subsystem exposes the animal to an undesirably high dose of X-rays to generate a three dimensional image.

For human patients, X-ray mammography is the modality of choice for breast cancer screening. However, the sensitivity of mammography is relatively low (between 70 and 80%), and the false positive rate is very high (between 70 and 90% of biopsies are normal). Conventional breast imaging is based on standard two dimensional (“2D”) X-ray mammography for screening and other modalities (ultrasound, MRI, or nuclear medicine) for diagnostic follow up. X-ray mammography may also be used for diagnostic follow up. Each modality has its unique strengths and weaknesses. For example, X-ray is typically used for detection characterization of microcalcifications and masses, while nuclear medicine can potentially provide differentiation between benign and malignant masses. However, combining (i.e., registering) the images obtained from X-ray and nuclear medicine mammography systems is very difficult since the x-ray exam is done with the breast compressed and the nuclear medicine exam is typically done by scanning an uncompressed breast.

### BRIEF SUMMARY OF THE INVENTION

In accordance with one preferred aspect of the present invention, there is provided a multi modality mammography imaging system, comprising a breast compression paddle, an X-ray mammography imaging subsystem adapted to image a breast compressed by the paddle, and a nuclear medicine mammography imaging subsystem adapted to image the breast compressed by the paddle.

In accordance with another preferred aspect of the present invention there is provided a multi modality imaging system, comprising an X-ray tomosynthesis subsystem and a nuclear medicine imaging subsystem.

In accordance with another preferred aspect of the present invention, there is provided a multi modality mammography imaging system, comprising a first means for compressing a patient's breast, a second means for X-ray imaging the breast compressed by the first means, and a third means for nuclear medicine imaging the breast compressed by the first means.

In accordance with another preferred aspect of the present invention, there is provided a nuclear medicine mammography system, comprising a breast compression paddle and a first nuclear medicine detector which is movably attached in or over the breast compression paddle.

In accordance with another preferred aspect of the present invention, there is provided a multi modality mammography method, comprising compressing a patient's breast, irradiating the compressed breast with X-rays, detecting the X-rays transmitted through the breast, acquiring at least one first data set of X-ray modality and forming a first image of the X-ray modality. The method also comprises detecting gamma rays emitted from the compressed breast, acquiring at least one second data set of nuclear medicine modality, forming a second image of the nuclear medicine modality, and co-registering the first and the second images.

### BRIEF DESCRIPTION OF THE DRAWINGS

FIG. 1 is a block diagram of a system according to preferred embodiments of the invention.

FIG. 2 is a block diagram of a subsystem according to the first preferred embodiment of the invention.

FIG. 3 is a three dimensional view of the subsystem according to the first preferred embodiment of the invention.

FIGS. 4-7 are schematic illustrations of the subsystem according to the first preferred embodiment of the invention.

FIG. 8 is a block diagram of a subsystem according to the second preferred embodiment of the invention.

FIG. 9 is a schematic top view of the subsystem according to the second preferred embodiment of the invention.

FIG. 10 is a schematic side cross sectional view of a nuclear medicine detector located in a breast compression paddle according to the second preferred embodiment of the invention.

### DETAILED DESCRIPTION OF THE INVENTION

The present inventors have realized that a multi modality system and method for mammography (i.e., human breast imaging) combining X-ray and nuclear medicine combines the strengths of two previously distinct modalities to address the limitations of today's breast imaging technologies. Specifically, in the multi modality mammography imaging system, a breast compression paddle is used to compress the breast during the X-ray and the nuclear medicine examinations. Thus, both the X-ray mammography imaging subsystem and the nuclear medicine mammography imaging subsystem of the system are adapted to image a breast compressed by the paddle. Breast compression for both modalities is preferably the same. However, compression may be reduced for the nuclear medicine imaging because it takes longer than X-ray imaging. Therefore, the ease of registration of the images obtained from X-ray and nuclear medicine mammography subsystems is improved since both the x-ray and the nuclear medicine exams are done with the breast compressed in the same position. The registered images may be fused to display a combined two or three dimensional image of the breast.

Imaging of a compressed breast provides significant advantages in mammography, both for x-ray and nuclear medicine imaging. Compression of the breast spreads and separates complex structures in the breast and reduces overall x-ray absorption, thus reducing the X-ray dose needed for imaging. The scintimammography images are also taken with the patient's breast compressed, to improve signal to noise ratio in the activity reconstructions (due to less gamma attenuation in the intervening breast tissue) and to improve the spatial resolution, since the collimator is closer to the breast in a compressed configuration. The use of breast compression for both modalities is also beneficial because the patient is immobilized and positioned in the same manner as with conventional X-ray mammography.

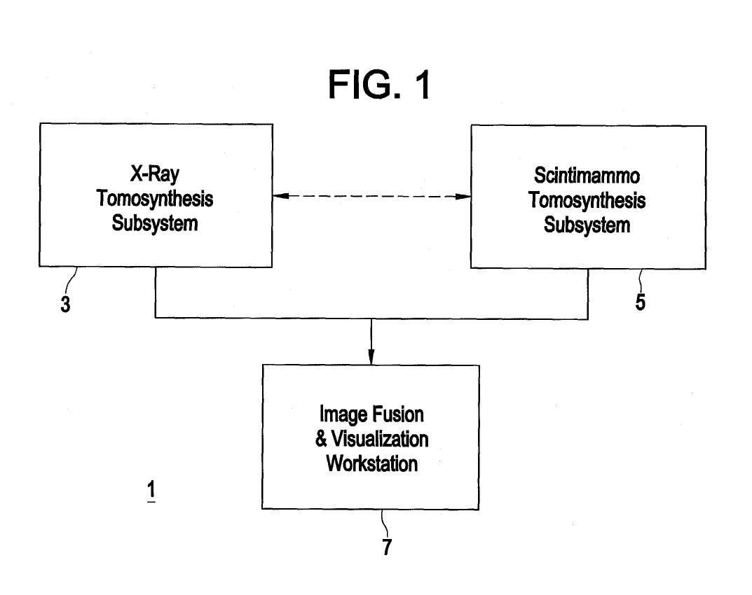

FIG. 1 illustrates a schematic of the multi modality imaging system 1. The system 1 includes an X-ray mammography imaging subsystem 3 and a nuclear medicine mammography imaging subsystem 5. These systems may optionally be directly electrically connected to share information, as indicated by the dashed line. The system 1 also contains an image fusion and visualization work station 7. This work station 7 may comprise a general or special purpose computer or any other type of image processor. The work station 7 receives data acquired by the subsystems 3 and 5 to form the image. Preferably, the work station 7 contains a processor which registers an X-ray image with a nuclear medicine image and a display with displays a fused X-ray and nuclear medicine image.

The X-ray mammography imaging subsystem 3 may comprise any X-ray imaging system, including a 2D X-ray mammography system which uses a digital detector, a 3D X-ray tomosynthesis system, in which the X-ray tube is scanned and a plurality of projection radiographs are acquired from different angles with respect to a stationary breast, or a 3D X-ray CT system in which the X-ray tube is angularly scanned 360 degrees. Likewise, the nuclear medicine mammography imaging subsystem 3 may comprise any nuclear medicine imaging system, including a 2D scintimammography system, a 3D scintimammo tomosynthesis system, a 3D positron emission mammography or a 3D nuclear medicine tomography system which uses 360 degree angular scanning, such as a SPECT system or a PET (“Positron Emission Tomography”) system. Any combination of the above subsystems may comprise the multi modality system 1, including 3D X-ray with 3D nuclear medicine, 3D X-ray with 2D nuclear medicine, 2D X-ray with 3D nuclear medicine, and 2D X-ray with 2D nuclear medicine.

In a first preferred embodiment of the present invention, the X-ray mammography imaging subsystem 3 comprises an X-ray tomosynthesis subsystem. In a second preferred embodiment of the present invention, the nuclear medicine imaging subsystem 5 comprises a scintimammo tomosynthesis subsystem. In a third preferred embodiment, both subsystems 3 and 5 comprise tomosynthesis subsystems which generate three dimensional X-ray and nuclear medicine images. These systems will now be described in detail.

The First Preferred Embodiment

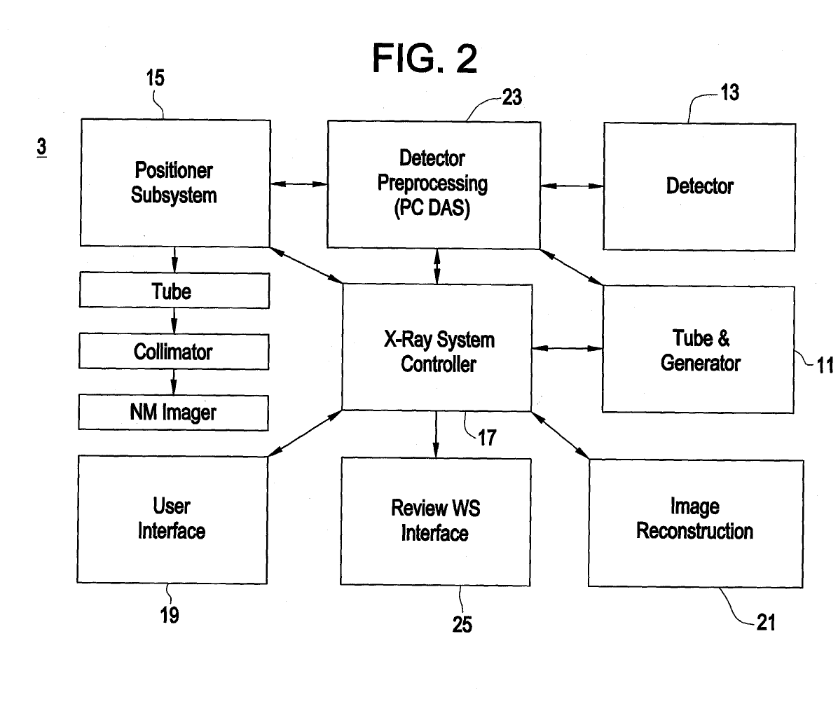

FIG. 2 is an illustration of the preferred components of the X-ray mammography tomosynthesis subsystem 3 of the first preferred embodiment of the present invention. It should be noted that the subsystem 3 may have additional components or lack one or more of the components described below. The subsystem contains an X-ray source 11, such as an X-ray tube and generator, and a detector 13, preferably a stationary digital detector. A positioner subsystem 15, such as a motor controller, is used to position the X-ray tube, the collimator and optionally the nuclear medicine detector, as will be described in more detail below.

The X-ray subsystem 3 also contains various electronic components. These components may comprise a single special or general purpose computer or a microprocessor chip, such as an ASIC chip. Alternatively, these electronic components may comprise several connected computers, processors or work stations. FIG. 2 illustrates how all of these components are interconnected. The electronic components include the X-ray subsystem controller 17, which controls the other electronic components, the positioner subsystem 15 and the X-ray source 11. The subsystem 3 also contains a user interface 19 and an image reconstruction section 21 which reconstructs a three dimensional image from two dimensional projection radiographs. A detector preprocessing and prefiltering section 23, such as a PC data acquisition subsystem, is connected to the detector 13. This section 23 removes artifacts, provides thickness compensation and data segmentation for the X-ray subsystem 3. Preferred reconstruction and preprocessing sections and methods are disclosed in related U.S. patent application Ser. No. 10/, (attorney docket number 040849/0186), to Jeffrey Eberhard and Bernhard Claus titled “Generalized Filtered Back-Projection Reconstruction In Digital Tomosynthesis” filed on the same date as the present application and incorporated herein by reference in its entirety.

The X-ray subsystem 3 also contains an optional review work station interface 25. This interface 25 is used to present to a clinician certain quantitative metrics extracted from the image. The clinician selects one or more metrics from a set of metrics to be computed and displayed on a workstation display or screen, whereby the metrics are displayed along with a mammographic image. For instance, interface 25 may be used to provide to the clinician access to 1) the overall percent glandular composition or 2) the percentage glandular distribution. Further, after delineation of findings (microcalcifications, masses, or vessels, e.g.), either via computer-aided diagnosis (CAD) algorithms or by hand-labeling, it may provide a summary of the quantitative measures of the findings. Such a preferred interface is disclosed in related U.S. patent application Ser. No. 10/, (attorney docket number 040849/0184), to John Kaufhold, Bernhard Claus and Jeffrey Eberhard titled “Method And Apparatus For Providing Mammographic Image Metrics To A Clinician” filed on the same date as the present application and incorporated herein by reference in its entirety.

The X-ray tomosynthesis subsystem 3 may have any desired physical layout. The tomosynthesis subsystem contains the X-ray source 11 adapted to move along a predefined trajectory, such as in an arc shaped path, a detector 13, such as a stationary digital X-ray detector, and a mechanical driving mechanism which is adapted to move the X-ray source along a predefined trajectory, such as the arc shaped path. Since the digital detector may be stationary while the X-ray source moves along the predefined trajectory, the tomosynthesis system provides multiple projection radiographs of the imaged breast from a single pass of the X-ray source through the predefined trajectory, from which a 3D representation of the imaged breast is reconstructed in the image reconstruction section 21. In contrast, in a CT system, the detector moves along with the X-ray source, and many X-ray shots are taken at a fine angular spacing as the X-ray source rotates 360 degrees around the object being imaged. Thus, a CT scan takes a longer time than a tomosynthesis scan and exposes the patient to a higher X-ray dose than a tomosynthesis scan. The tomosynthesis system is advantageous in that it uses a total X-ray dose to form a three dimensional image that is about the same as a dose required to form a single X-ray projection image. The low X-ray dose required to form a three dimensional image in a X-ray tomosynthesis system is especially advantageous when used in combination with the nuclear medicine detector. In nuclear medicine, the patient is provided with a radioactive material. Thus, it is desirable to decrease the X-ray dose as much as possible when providing the patient with the radioactive material.

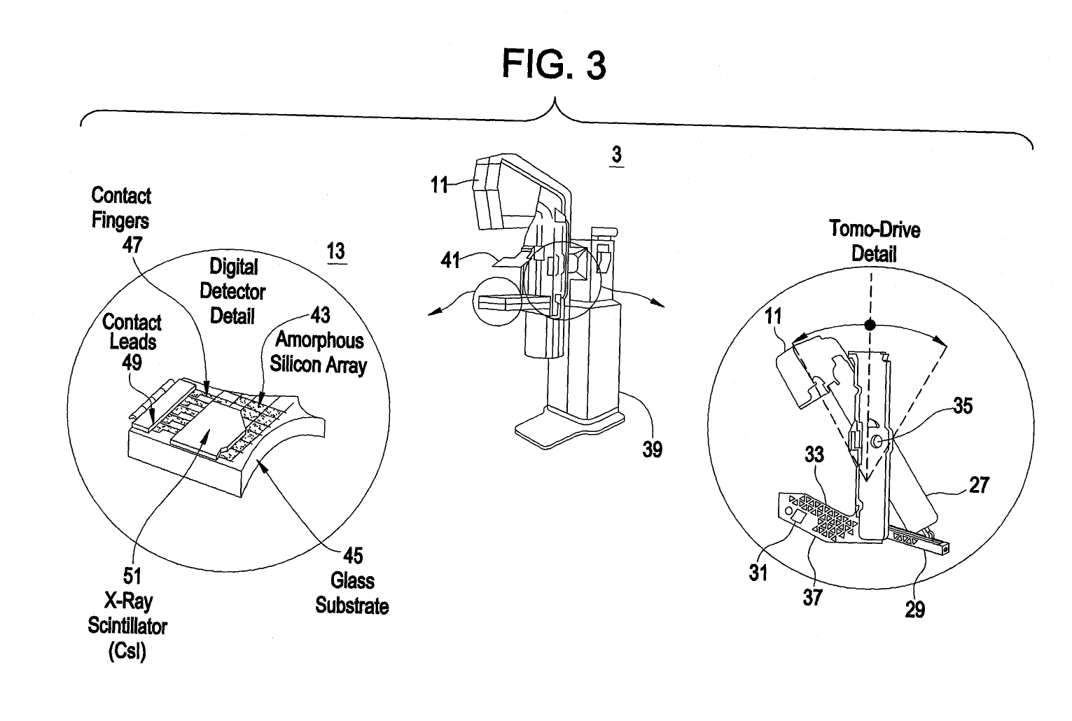

FIG. 3 illustrates one layout of the X-ray tomosynthesis subsystem 3 with a track for moving the X-ray source according to the first preferred embodiment. This system is described in detail in related U.S. patent application Ser. No. 10/, (attorney docket number 040849/0187), to Yu Wang, Reinhold Wirth and James Alexander titled “Tomosynthesis X-Ray Mammogram System And Method With Automatic Drive System” filed on the same date as the present application and incorporated herein by reference in its entirety.

The X-ray source 11 is mounted to an upper or first portion of the first arm 27. The first arm 27 may have any desired shape, such as a tube or plate shape. A lower or second portion of the first arm 27 distal from the first portion is mounted to a linear motion track 29.

The mechanical driving mechanism, such as a ball screw driven by a motor (not shown in the figure because the ball screw is located in the track) is adapted to move the lower portion of the first arm 27 along the track 29, to move the X-ray source 11 in the arc shaped path. The motor may also be mounted onto the track if desired. A side pin 31 is positioned to create a stable whole range drive by allowing the track 29 to rotate with respect to a fixed point.

The detector 13 is mounted to a second support or arm 33. Typical detector size for X-ray acquisition is 24 cm×30 cm or 18 cm×24 cm. However, other suitable dimensions may be used. The second arm 33 may have any desired shape, such as a tube or plate shape. A shaft 35 connects the middle portions of the first arm 27 and the second arm 33, such that the arms 27, 33 may rotate relative to each other about the shaft 35 in a scissors-like motion. Preferably, the second arm 33 is stationary while the first arm 27 rotates.

In a preferred aspect of the first embodiment, a pivot point plate 37 is attached to the second arm 33, as shown in FIG. 3. The pivot point plate 37 is rotatably mounted to the linear motion track 29 by the side pin 31. The pivot plate 37 and track 29 optionally have holes which reduce the weight of the plate and track. While the second arm 33 supporting the detector 13 and the pivot plate 37 remain stationary, the first arm 27 rotates and the track 29 moves in a vertical plane with respect to the second arm 33 about the side pin 31. The combined motion of the first arm 27 and the track 29 allows the first arm to move along a linear motion track 29 while moving the X-ray source 11 in an arc shaped path.

The X-ray tomosynthesis subsystem 3 is mounted to a gantry or base 39. The detector 11 is mounted over the gantry 39 in a position which allows a patient to place her breast onto the detector. The subsystem 3 may be adjustable in the vertical direction relative to the ground to allow patients of different height to use the system without stretching or bending. The compression paddle 41 is likewise height adjustable. The preferred electronic detector 13 contains an amorphous silicon photodetector array 43 formed on a glass substrate 45. The array 43 includes metal contact fingers 47 and metal contact leads 49. An X-ray sensitive scintillator material 51, such as cesium iodide, is formed over the array 43. The scintillator material 51 emits radiation having a wavelength detectable by the silicon pixels in the array 43 in response to receiving an X-ray. However, various other solid state and vacuum digital X-ray detectors may be used instead. The magnitude of the radiation is a function of the attenuation of the X-rays by the imaged object. The pixels of array 43 convert the received radiation into an electrical signal of a predetermined magnitude that is provided to the preprocessor 23 and then converted into an image.

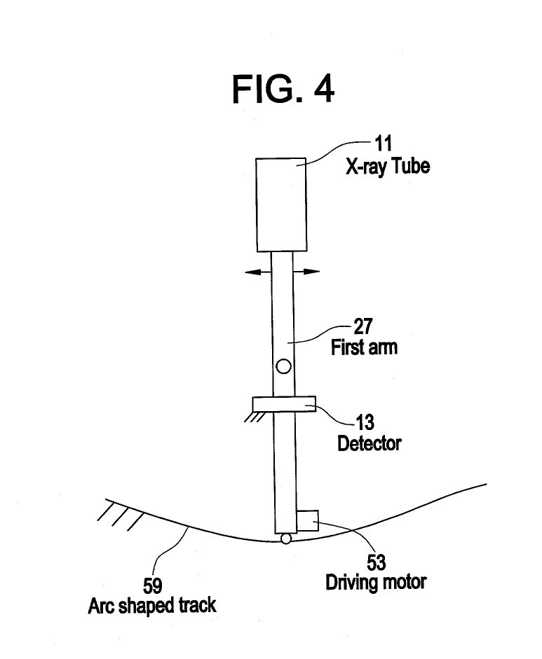

However, in alternative preferred aspects of the X-ray tomosynthesis subsystem 3, an arc shaped track is used instead of a linear motion track. For example, in a second preferred aspect, an arc shaped track 59 is used instead of a linear motion track 29, as schematically illustrated in FIG. 4. The lower portion of the first arm 27 is moved along the track 59 by a motor 53. This causes the X-ray source 11 supported by the upper portion of the first arm 27 to move in an arc shaped path.

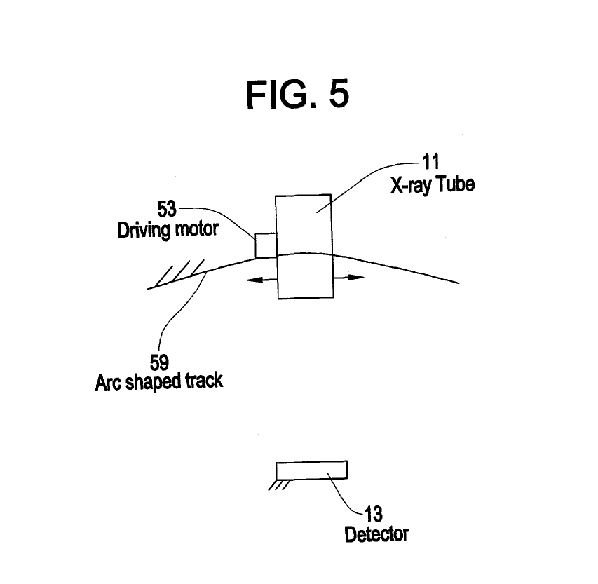

The subsystem 3 of the third preferred aspect is schematically illustrated in FIG. 5. In this embodiment, the X-ray source 11 is mounted directly to the arc shaped track 59. The motor 53 is attached to the X-ray source 11 and is adapted to move the X-ray source along the arc shaped track 59. The motor 53 is also preferably attached to the track 59. The digital detector 13 is located facing the X-ray source 11 such that an imaging area is formed above the detector. In this embodiment, the first arm 27 may be omitted.

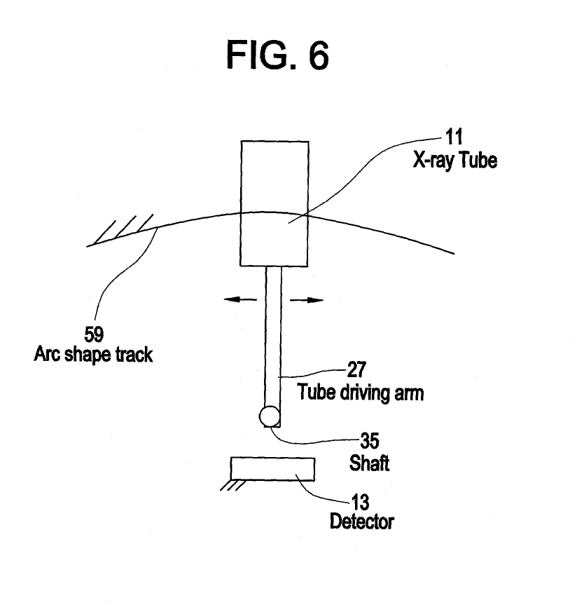

The subsystem 3 of the fourth preferred aspect is schematically illustrated in FIG. 6. In this embodiment, the X-ray source 11 is also mounted to the arc shaped track 59. However, the first arm 27 is used to move the X-ray source 11 in the arc shaped path. Preferably, the first arm 27 is made relatively thin and light weight to minimize its mass, but has sufficient rigidity to move the X-ray source 11 along the track 59. The first arm 27 connects the X-ray source 11 to the shaft 35. The shaft 35 connects the first arm 27 to the second arm 33 supporting the detector 13. The shaft 35 is turned by a motor or other rotation imparting device (not shown). The step motion of the X-ray source 11 is produced from the shaft 35 torque through the first arm 27. Since a track is used to move the X-ray source 11 in the four above described aspects, the X-ray source 11 motion is precisely controlled by the track. This reduces the system vibration and improves the image quality.

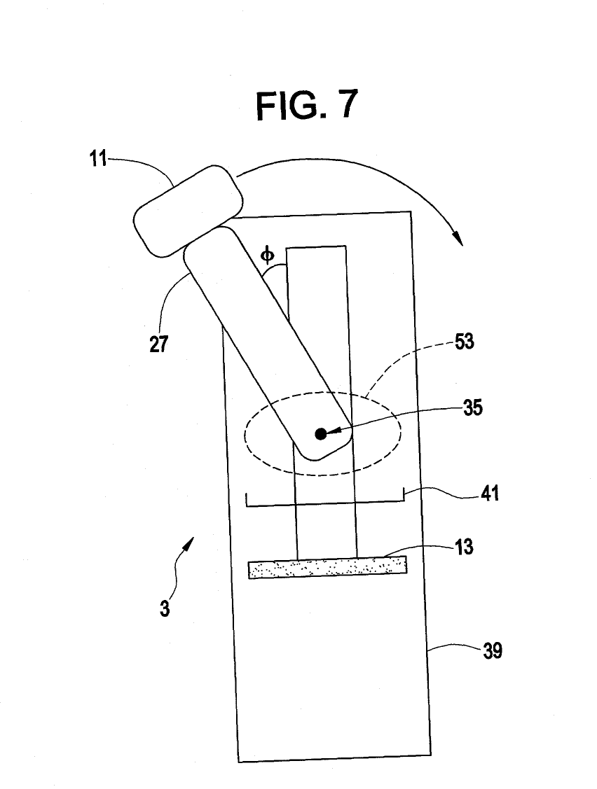

The subsystem 3 of the fifth preferred aspect is schematically illustrated in FIG. 7. This subsystem 3 shown in FIG. 7 is disclosed in U.S. Pat. No. 5,872,828, incorporated herein by reference in its entirety. The detector 13 is mounted on a stationary portion of the gantry 39. The X-ray source 11 is mounted onto an upper portion of a movable arm 27. The lower end of the arm 27 is pivotably attached to the gantry 39. As shown in FIG. 7, the X-ray source 11 pivots from arm 27 about a point 35 (such as a shaft) above the detector 13. The x-ray source 11 is stationary during the exposure and then is moved to the next position in its arc shaped path before obtaining the next image. An actuator or control mechanism 53 is used to rotate the arm 27 any angle up to +/−27 degrees from a direction perpendicular to the detector 13.

The X-ray tomosynthesis method includes irradiating the compressed breast with X-rays. Preferably the X-ray source is mechanically moved using a track in a stepped motion on the arc shaped path around the compressed breast. The compressed breast is irradiated with an X-ray dose from the X-ray source located at a plurality of steps along the arc shaped path. The X-rays transmitted through the breast are detected with a stationary digital X-ray detector. A three dimensional image of the X-ray modality is constructed from a signal output by the digital X-ray detector.

The multi modality imaging system 1 which includes the X-ray tomosynthesis subsystem 3 described above used in combination with any nuclear medicine imaging subsystem 5 is preferably used for mammography. However, multi modality imaging system 1 which includes the X-ray tomosynthesis subsystem 3 and the nuclear medicine imaging subsystem 5 may be used to image any other part of a human body in addition to the breast, as well as to image animals, if desired.

Furthermore, the multi modality imaging system 1 is not limited to only two modalities. For example, if desired, a third modality, such as an ultrasound modality, may be added to the system 1. Thus, an ultrasound imaging subsystem may be added to the system 1. An imaging system which incorporates an X-ray tomosynthesis subsystem and an ultrasound subsystem is described in a commonly assigned, copending U.S. patent application Ser. No. 10/ ______ (attorney docket number RD 29,241) titled “Methods, System and Apparatus For Digital Imaging” to Ajay Kapur, et al., filed on Feb. 1, 2002 and incorporated herein by reference in its entirety.

The Second Preferred Embodiment

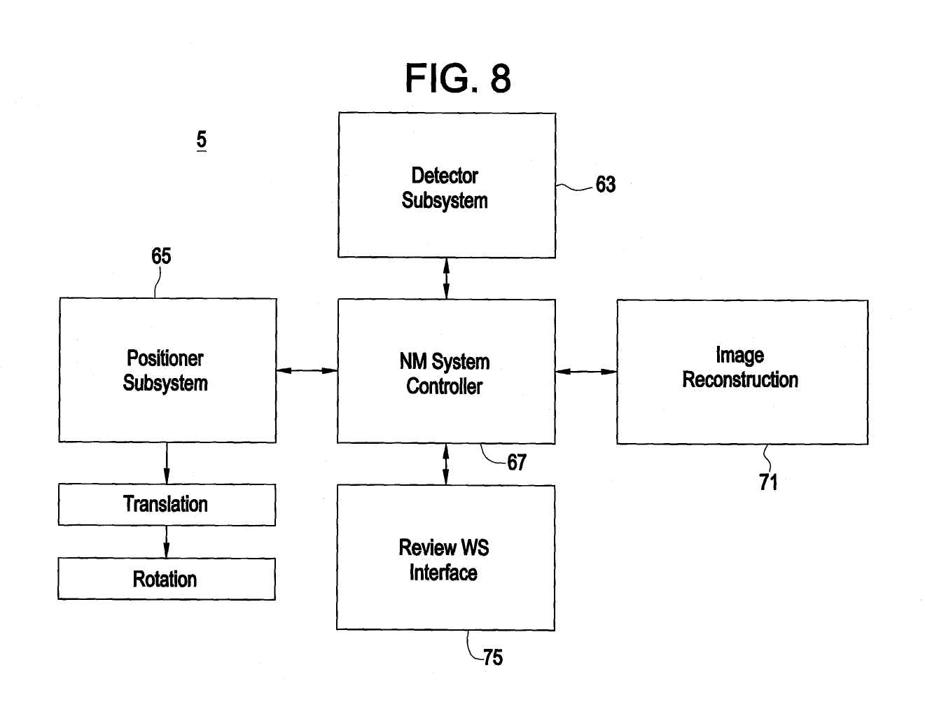

FIG. 8 is an illustration of the preferred components of the scintimammo tomosynthesis subsystem 5 of the second preferred embodiment of the present invention. It should be noted that the subsystem 5 may have additional components or lack one or more of the components described below.

The nuclear medicine subsystem 5 contains a nuclear medicine detector subsystem 63. The detector subsystem 63 may comprise one or more nuclear medicine detectors. A nuclear medicine detector preferably includes a gamma ray sensitive scintillator or phosphor and a position sensitive radiation detector. The radiation detector may be any detector which detects the radiation emitted by a scintillator or phosphor pixel in response to a gamma ray striking this pixel. For example, the detector may comprise a solid state detector array, such as a semiconductor photodiode or charge coupled device array, or a vacuum position sensitive radiation detector, such as a position sensitive photomultiplier tube.

The nuclear medicine subsystem 5 also contains a positioner subsystem 65, such as a motor controller. The positioner subsystem 65 is used to position one or more nuclear medicine detectors 63 by rotation and/or translation of the detectors 63, as will be described in more detail below. The positioner subsystem 65 may be the same or a different subsystem from the X-ray positioner subsystem 15.

The nuclear medicine subsystem 5 contains various electronic components. These components may comprise a single special or general purpose computer or a microprocessor chip, such as an ASIC chip. Alternatively, these electronic components may comprise several connected computers, processors or work stations. The nuclear medicine subsystem 5 electronic components may be the same components as in the X-ray subsystem 3 (i.e., the electronics are shared between the X-ray and nuclear medicine subsystems) or some or all of the electronic components may comprise different components from those in the X-ray subsystem 3 (i.e., the electronic components are not shared between subsystems 3 and 5). FIG. 8 illustrates how all of these components are interconnected.

The electronic components include the nuclear medicine subsystem controller 67, which controls the other electronic components and the positioner subsystem 65. The subsystem 5 also contains an image reconstruction section 71 which reconstructs a three dimensional image from two dimensional images. The nuclear medicine subsystem 5 also contains an optional review work station interface 75. This interface 75 is used to display the nuclear medicine image and to optionally present to a clinician certain quantitative metrics extracted from the image. The clinician selects one or more metrics from a set of metrics to be computed and displayed on a workstation display or screen, whereby the metrics are displayed along with a mammographic image.

The nuclear medicine detector(s) may be arranged in any desired configuration suitable for a particular nuclear medicine subsystem 5. Preferably, the nuclear medicine detector(s) 63 is arranged for a partial rotation about the breast in a scintimammo tomosynthesis nuclear medicine subsystem 5 which is based on the single photon emission principle. Alternatively, at least two oppositely positioned detectors may be used for a positron emission mammography nuclear medicine subsystem or for a PET nuclear medicine subsystem. Furthermore, a stationary or rotating ring type detector may be used for a PET or SPECT nuclear medicine subsystem.

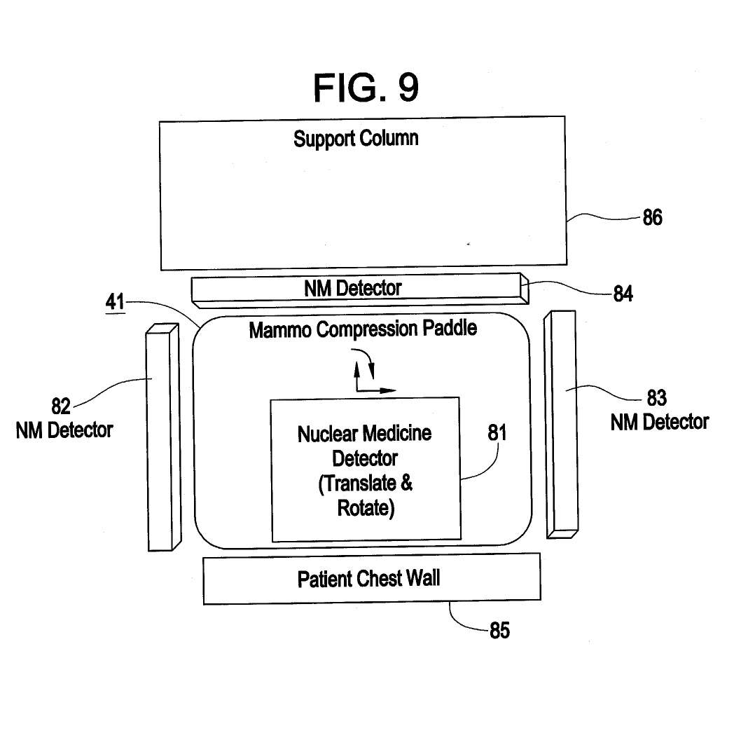

FIG. 9 illustrates a top view of the preferred arrangement of nuclear medicine detectors in the system 1 for use with a scintimammography tomosynthesis nuclear medicine subsystem 5. Preferably, a first nuclear medicine detector 81 is movably and removably attached in or over the breast compression paddle 41 of the system 1. Most preferably, the detector 81 is movably attached over the paddle 41, with the gamma ray sensitive sctintillator (or phosphor) facing the paddle. It should be noted that “over the paddle” is a relative term, which means that the detector is located on the opposite side of the paddle from the breast in configurations where the subsystem 5 is positioned non-vertically or if the paddle 41 compresses the breast from below or from the side. Alternatively, the nuclear medicine detector 81 can be placed within the compression paddle 41 to assure that it is as close to the breast as possible. The term “within” includes the configuration where the detector 81 itself is used as a compression paddle 41 to compress the breast.

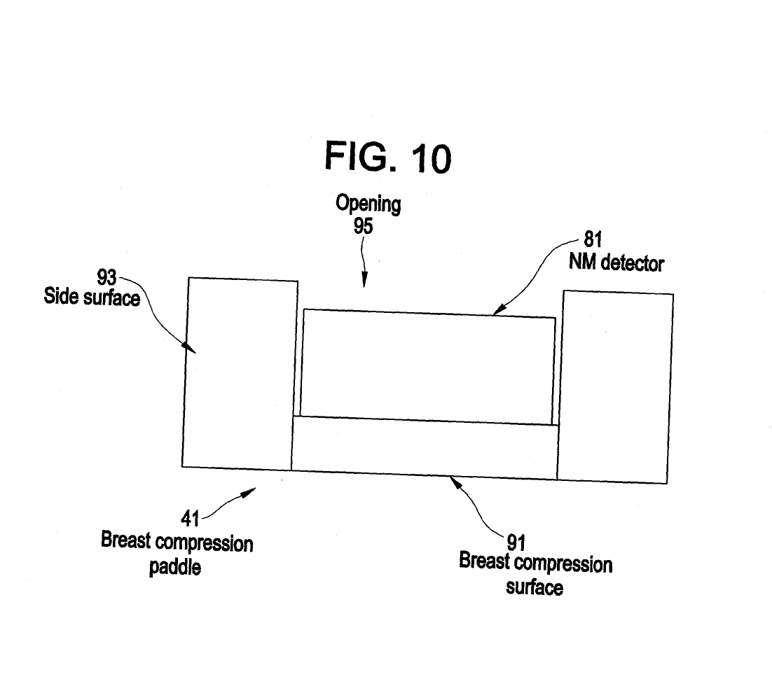

FIG. 10 illustrates a side cross sectional view of one preferred configuration of the paddle 41 where the first nuclear medicine detector 81 is located in the paddle 41. The paddle 41 contains a breast compression surface 91. For a horizontally positioned paddle, surface 91 is preferably the lower surface of the paddle. The paddle also preferably contains one or more side surfaces 93, which add structural rigidity to the paddle. For a horizontally positioned paddle, surfaces 93 preferably extend upwards from the lower surface 91 of the paddle. The side surfaces 93 delineate a opening 95 over the breast compression surface 91. The first nuclear medicine detector 81 is removably positioned in the opening 95 between the side surfaces 93 on the breast compression surface 91, such that the breast compression surface 93 is located between the breast and the detector 81. Other paddle 41 configurations may be used, if desired.

This favorable geometry improves the spatial resolution of the nuclear medicine imaging subsystem 5. The paddle 41 is made of a material which allows gamma rays to pass through it, such as a plastic or polymer material (i.e., polycarbonate, polystyrene, PMMA, epoxy, etc.). However, other paddle 41 materials may be used if desired.

The positioner subsystem or assembly 65 is used to translate (i.e., move the detector 81 in a plane substantially parallel to the paddle) and rotate the first nuclear medicine detector 81 relative to the breast compression paddle 41. The positioner subsystem 65 may comprise one or more motors, such as DC motors, stepper motors, etc., which move the detector 81 along X and Y carriages over or in the paddle 41. Other suitable positioner 65 configurations may be used if desired. The carriages may be mounted onto a frame, such as a U-shaped frame, for support. The nuclear medicine mammography system and method with a breast compression paddle and a nuclear medicine detector in or above the paddle may be used alone without the X-ray imaging system or in combination with other modalities, such as ultrasound.

Preferably, the nuclear medicine subsystem 5 also contains a decoupling assembly (not shown in FIG. 9 for clarity). The decoupling assembly allows the first nuclear medicine detector 81 to be removed from over the paddle 41 or from inside the paddle 41. For example, the decoupling assembly may comprise a slide adapter containing rails or carriages. This assembly allows the first detector 81, alone or together with the positioner subsystem 65, to be moved away from the paddle 41 during the X-ray mammography step, such that the detector 81 does not interfere with the X-rays emitted by the X-ray source 11. Alternatively, the decoupling assembly may have another configuration, such as a movable arm which supports the detector 81 and/or the positioner subsystem 65, and may be swung out of the way during X-ray mammography. The detector 81 and/or the positioner subsystem 65 may be moved away from the paddle either manually or mechanically using the decoupling assembly.

In a preferred aspect of the second embodiment, the subsystem 5 contains at least one additional nuclear medicine detector located in a plane substantially perpendicular to the plane of the compression paddle 41. For example, there may be two nuclear medicine detectors 82 and 83 located on opposite sides of the detection volume below the breast compression paddle 41, as shown in FIG. 9. The detection volume is located directly below the paddle 41 where the patient's breast is located. If desired, there may be an optional third nuclear medicine detector 84. The detector 84 is located in a plane substantially perpendicular to the plane of the compression paddle 41 and on an opposite side of the imaging volume from the position of the patient chest wall 85. This third detector 84 may be located adjacent to the support column 86 or gantry of the system 1. A plane “substantially perpendicular” to the paddle 41 includes vertical planes and planes which deviate by about 15 degrees or less from the vertical direction, if the paddle 41 is positioned in a horizontal plane. However, these planes would be different if the paddle 41 is positioned in a non-horizontal plane.

Additional detectors 82, 83, 84 located on the sides of the breast (i.e., on the sides of the detection volume) provide useful depth information. If desired, these detectors may have smaller collimator apertures to overcome resolution loss due to their greater distance from the activity concentrations. The effect of the smaller apertures on signal level and hence on signal to noise ratio is compensated by the fact that these detectors can be active for all positions of the angular scan of the first nuclear medicine detector 81 above or in the compression paddle. Hence the data acquisition time may be substantially longer. In addition, the use of multiple detectors 82, 83, 84 (one detector on each of the 3 “non-chest wall” sides of the breast) enhances total number of counts and hence signal level and signal to noise ratio. A non-scanning nuclear medicine acquisition configuration is based on one detector 81 above the compression paddle and one or more detectors 82, 83 and/or 84 on the “non-chest wall” sides of the breast, as described above.

For nuclear medicine breast imaging, various sizes of nuclear medicine detectors may be used, such as 5 cm×5 cm, 10 cm×10 cm, 15 cm×20 cm, or any other desired dimensions. Smaller nuclear medicine detectors are beneficial because they can be positioned closer to the compression paddle 41 for a large range of scanning angles, but they limit the field of view of coverage. Furthermore, for breast imaging, isotopes with lower energy gamma ray could be used, since the geometry is favorable.

The nuclear medicine imaging method includes detecting gamma rays emitted from the compressed breast. Preferably, gamma rays which pass through a breast compression paddle are detected with a nuclear medicine detector. A nuclear medicine modality image is then formed. Preferably, a three dimensional image of the breast is formed from a signal output by the nuclear medicine detector.

Since the nuclear medicine detector 81 is removable, the X-ray imaging is preferably performed first, before the detector 81 detector is put in place. The nuclear medicine detector 81 is then positioned over a region of interest and rotated through multiple angles to acquire data at various angular positions relative to the breast. The preceding x-ray acquisition is optionally used to properly position the detector 81 over the field of view of interest, if a full field nuclear medicine detector is not used. As in X-ray tomosynthesis, this acquisition at multiple angles allows for depth resolution in the z-direction. The angular scanning range can be modified depending on the position of the region of interest. For example, for a lesion near the edge of the breast, the lesion may be outside the field of view for roughly one half the normal scanning angles. These views could therefore be eliminated and more time spent on the views which provide useful information.

Conventional nuclear medicine reconstruction algorithms can be used, suitably modified for incomplete data acquisition. Alternatively, tomosynthesis reconstruction algorithms, with a correction for attenuation in intervening breast tissue, can be used. Attenuation values can be derived directly from the tomosynthesis images. This type of algorithm has the advantage that artifact reduction due to limited angle acquisition is incorporated directly into the reconstruction.

In an alternative aspect of the second preferred embodiment, positron emission mammography imaging is performed. In this aspect, detectors on both sides of the breast are used for coincidence detection. This is possible in a magnification geometry where the breast is compressed on a “mag stand” positioned above the image receptor. However, scintimammography provides reduced system complexity and cost compared to positron emission imaging (one detector instead of two, no complex coincidence circuitry, positioning ease, etc.).

The Third Preferred Embodiment A preferred multi modality mammography method using the X-ray and nuclear medicine system 1 will now be described. The method generally includes compressing a patient's breast, such as with the paddle 41, irradiating the compressed breast with X-rays, such as from the X-ray source 11, detecting the X-rays transmitted through the breast, such as with detector 13, acquiring at least one first data set of X-ray modality and forming a first image of the X-ray modality using the electronics illustrated in FIG. 2. The method also includes detecting gamma rays emitted from the compressed breast, such as with one or more nuclear medicine detectors 81-84, acquiring at least one second data set of nuclear medicine modality and forming a second image of the nuclear medicine modality using the electronics illustrated in FIG. 8. Preferably, at least one of, and more preferably both of the first and the second data sets comprise a three dimensional data set. Preferably, at least one of, and more preferably both of the first and the second images comprise a three dimensional image.

The first and the second images are then co-registered by the electronics illustrated in FIGS. 2 and 8. Preferably, the first image and the second image are fused to form a composite three dimensional image and the fused image is displayed using the electronics illustrated in FIGS. 2 and 8. However, the co-registered images may be displayed side by side rather than fused, if desired. Furthermore, the image(s) may be stored or transmitted to a remote location rather than being displayed.

Thus, the data sets from multiple imaging modalities are acquired, images from each modality are reconstructed and displayed, and the multi-modality images are jointly visualized. Data sets from two or more modalities can be fused. Information from one modality can be used to enhance the acquisition, image reconstruction or display of the other. Fusion is based on mechanically co-registered acquisition, co-registered acquisition supplemented by imaging physics (i.e., knowledge of energy propagation paths for the various modalities), mutual information based registration, or other registration methods.

The preferred image processing method includes the following steps. Data sets of the first modality are acquired. This includes one or more data sets at various orientations of the sensor and/or radiation source with respect to the breast. Images of the first modality are then formed. Optionally, additional information from other modalities is used to optimize image quality. The images of the first modality are displayed and visualized. These steps are then repeated for the second modality. If additional modalities are present, then the process is repeated for these additional modalities. This is followed by co-registration, fusion, and co-registered display of the multi-modality images. Preferably, the nuclear medicine detector 81 is moved away from the breast compression paddle 41 prior to the step of irradiating the compressed breast with X-rays. Then, the nuclear medicine detector 81 is moved back into or over the breast compression paddle 41 prior to the step of the step of detecting gamma rays.

In the preferred embodiment of X-ray/scintimammography fusion, the preferred method is to acquire x-ray data for tomosynthesis, to create a 3D image of X-ray attenuation using a reconstruction algorithm, and then to visualize the 3D images using volume rendering or cine mode display. Then, scintimammography data is acquired over a similar angular range (with a similar or different number of acquisition positions, depending on the acquisition timing requirements). A 3D image of radioactive pharmaceutical uptake activity is created using a reconstruction algorithm, and the 3D images are visualized using volume rendering or cine mode display. The data sets acquired from the two modalities are co-registered geometrically, so the relative size and orientation of the data sets are known by position of the acquisition sensors. The physics of the imaging configuration can be used to improve registration and correct for known propagation effects. Finally, mutual information registration techniques can be used to enhance information fusion.

The detectors transmit data regarding projection radiographs which form a projection image or a “view.” Then a collection or plurality of views (a projection data set) is used to reconstruct image “slices” (reconstructed cross-sectional images representative of the structures within the imaged object at a fixed height in a plane parallel to the detector surface) or reconstruction planes (reconstructed cross-sectional images representative of the structures within the imaged object at a fixed height in a plane not parallel to the detector surface). A collection or a plurality of slices and/or reconstruction planes for all heights (three-dimensional dataset representative of the imaged object) is then used to reconstruct a three dimensional image.

A computer aided detection method may be used for detecting a region of concern in at least one of a first image of the breast generated by a first modality and a second image of the breast generated by a second modality. The detected region of concern is classified, correlated with a corresponding region in the other one of the first image and the second image, and the classification is weighted with a weighting factor corresponding to a degree of correlation. This method is described in detail in related U.S. patent application Ser. No. 10/, (attorney docket number 040849/0181), titled “Computer Aided Detection (CAD) For 3D Digital Mammography” to Jeffrey Eberhard, Abdalmajeid Alyassin and Ajay Kapur, filed on the same date as the present application and incorporated herein by reference in its entirety.

A typical range of angular scanning is +/−45 degrees, preferably +/−25 degrees around the axis perpendicular to the detector surface. This configuration allows the standard breast compression geometry to be used, which simplifies patient positioning and radiologist familiarity with image format. However, other ranges are possible, all the way from a single view acquisition for each modality (fusion of two dimensional x-ray mammography and scintimammography) to full 360 degree angular scanning (such as computed tomography (CT) geometry for X-ray and SPECT or PET geometry for nuclear medicine). Thus, four general categories of fusion are possible: 3D X-ray with 3D nuclear medicine; 3D X-ray with 2D nuclear medicine; 2D X-ray with 3D nuclear medicine; and 2D X-ray with 2D nuclear medicine. The method can be generalized to co-registered acquisition of other breast imaging modalities, including fusion of more than 2 modalities (such as x-ray, nuclear medicine, and ultrasound), if clinical requirements demand it.

Since the X-ray and nuclear medicine data are acquired in the same physical configuration of the breast, the images can be registered directly from the mechanical registration information. Alternately, the physics of the individual imaging modalities can be used to enhance the registration of the two images. Differences in spatial resolution in the two modalities, and in propagation characteristics can be taken into account to identify small positioning differences in the two images. Registration is then based on corrected positions in the 3D data sets, where the corrections are based on the imaging physics of the two modalities (such as knowledge of energy propagation paths for the various modalities). In addition, feature based registration can be used to identify structures in one image and find corresponding structures in the other modality image. The data sets can be registered and displayed to capture the corresponding information from both images simultaneously for evaluation by the radiologist.

The amount of breast compression for both X-ray and nuclear medicine modalities may be slightly less than in conventional X-ray mammography, if three dimensional (“3D”) tomosynthesis imaging is used, because it reduces effects of superimposed tissue on suspicious regions in the breast. The reduced compression in tomosynthesis results in an increase in patient comfort, which is advantageous because the scintimammography scan may take many minutes to accomplish.

The preferred embodiments have been set forth herein for the purpose of illustration. However, this description should not be deemed to be a limitation on the scope of the invention. Accordingly, various modifications, adaptations, and alternatives may occur to one skilled in the art without departing from the scope of the claimed inventive concept.

What is claimed is:

1. A multi modality mammography imaging system, comprising:
a breast compression paddle;
an X-ray mammography imaging subsystem adapted to image a breast compressed by the paddle; and
a nuclear medicine mammography imaging subsystem adapted to image the breast compressed by the paddle.

2. The system of claim 1, wherein:
the X-ray mammography imaging subsystem comprises an X-ray mammography tomosynthesis subsystem; and
the nuclear medicine mammography imaging subsystem comprises a scintimammo tomosynthesis subsystem.

3. The system of claim 2, further comprising:
a processor which registers an X-ray image with a nuclear medicine image; and
a display which displays a fused X-ray and nuclear medicine image.

4. The system of claim 2, wherein the X-ray mammography tomosynthesis subsystem comprises:
an X-ray source adapted to move in an arc shaped path;
a stationary digital X-ray detector; and
a mechanical driving mechanism which is adapted to move the X-ray source in the arc shaped path.

5. The system of claim 4, wherein the X-ray mammography tomosynthesis subsystem further comprises a track which is used to move the X-ray source in the arc shaped path.

6. The system of claim 2, wherein the scintimammo tomosynthesis subsystem comprises a first nuclear medicine detector located in or over the breast compression paddle.

7. The system of claim 6, further comprising at least one second nuclear medicine detector located in a plane substantially perpendicular to the plane of the compression paddle.

8. The system of claim 6, wherein the first nuclear medicine detector is removably and rotatably attached in or over the breast compression paddle.

9. A multi modality imaging system, comprising:
an X-ray tomosynthesis subsystem; and
a nuclear medicine imaging subsystem.

10. The system of claim 9, wherein:
the X-ray tomosynthesis subsystem comprises an X-ray mammography tomosynthesis subsystem; and
the nuclear medicine imaging subsystem comprises a scintimammo tomosynthesis, a positron emission mammography, SPECT or PET subsystem.

11. The system of claim 10, further comprising:
a breast compression paddle;
a processor which registers an X-ray image with a nuclear medicine image; and
a display which displays a fused X-ray and nuclear medicine image.

12. The system of claim 11, wherein the X-ray mammography tomosynthesis subsystem comprises:
an X-ray source adapted to move in an arc shaped path;
a stationary digital X-ray detector; and
a mechanical driving mechanism which is adapted to move the X-ray source in the arc shaped path.

13. The system of claim 12, wherein the X-ray mammography tomosynthesis subsystem further comprises a track which is used to move the X-ray source in the arc shaped path.

14. The system of claim 11, wherein the nuclear medicine imaging subsystem comprises a scintimammo tomosynthesis subsystem having a first nuclear medicine detector located in or over the breast compression paddle.

15. The system of claim 14, further comprising at least one second nuclear medicine detector located in a plane substantially perpendicular to the plane of the compression paddle.

16. The system of claim 15, wherein the first nuclear medicine detector is removably and rotatably attached in or over the breast compression paddle.

17. The system of claim 9, further comprising an integrated ultrasound imaging subsystem.

18. A multi modality mammography imaging system, comprising:
a first means for compressing a patient's breast;
a second means for X-ray imaging the breast compressed by the first means; and
a third means for nuclear medicine imaging the breast compressed by the first means.

19. The system of claim 18, further comprising:
a third means for registering a three dimensional X-ray image with a three dimensional nuclear medicine image; and
a fourth means for displaying a three dimensional fused X-ray and nuclear medicine image.

20. The system of claim 19, wherein the second means comprises:
a fifth means for irradiating the breast with an X-ray dose at a plurality of steps along an arc shaped path;
a sixth means for mechanically moving the fifth means in a stepped motion on the arc shaped path around the breast; and
a seventh means for detecting the X-rays transmitted through the breast.

21. The system of claim 19, wherein the third means comprises an eighth means for detecting gamma rays located in or over the first means.

22. The system of claim 21, further comprising at least one ninth means for detecting gamma rays located in a plane substantially perpendicular to the plane of the compression paddle.

23. A nuclear medicine mammography system, comprising:
a breast compression paddle; and
a first nuclear medicine detector which is movably attached in or over the breast compression paddle.

24. The system of claim 23, wherein the first nuclear medicine detector is located in the breast compression paddle.

25. The system of claim 23, further comprising:
a positioner assembly adapted to translate and rotate the first nuclear medicine detector relative to the breast compression paddle; and
a decoupling assembly which allows the first nuclear medicine detector to be removed from over or inside the breast compression paddle.

26. The system of claim 25, wherein:
the nuclear medicine mammography system comprises a scintimammography tomosynthesis system; and
the first nuclear medicine detector comprises a gamma ray sensitive scintillator optically coupled to a solid state photo detector or to a photomultiplier tube.

27. The system of claim 26, further comprising a processor which is adapted to generate a three dimensional nuclear medicine image of the breast.

28. The system of claim 23, further comprising at least one second nuclear medicine detector located in a plane substantially perpendicular to the plane of the compression paddle.

29. The system of claim 28, wherein the at least one second nuclear medicine detector comprises two nuclear medicine detectors located on opposite sides of a detection volume below the breast compression paddle.

30. The system of claim 29, further comprising a third nuclear medicine detector located in a plane substantially perpendicular to the plane of the compression paddle and on an opposite side of the imaging volume from a patient position.

31. The system of claim 23, further comprising:
an X-ray source; and
an X-ray detector.

32. The system of claim 31, wherein:
the X-ray source and the X-ray detector comprise an X-ray mammography tomosynthesis subsystem;
the X-ray source is adapted to be moved in an arc shaped path above the breast compression paddle by a mechanical driving mechanism; and
the X-ray detector is a stationary digital X-ray detector which is located across the imaging volume from the breast compression paddle.

33. A multi modality mammography method, comprising:
compressing a patient's breast;
irradiating the compressed breast with X-rays;
detecting the X-rays transmitted through the breast;
acquiring at least one first data set of X-ray modality;
forming a first image of the X-ray modality;
detecting gamma rays emitted from the compressed breast;
acquiring at least one second data set of nuclear medicine modality;
forming a second image of the nuclear medicine modality; and
co-registering the first and the second images.

34. The method of claim 33, wherein:
at least one of the first and the second data sets comprises a three dimensional data set; and
at least one of the first and the second images comprises a three dimensional image.

35. The method of claim 34, wherein:
the first and the second data sets comprise three dimensional data sets; and
the first and the second images comprise a three dimensional image.

36. The method of claim 35, wherein:
a plurality of first data sets are acquired at a plurality of orientations of at least one of an X-ray source and an X-ray detector; and
a plurality of second data sets are acquired at a plurality of orientations of a nuclear medicine detector.

37. The method of claim 36, wherein:
the step of irradiating the compressed breast with X-rays comprises mechanically moving an X-ray source using a track in a stepped motion on an arc shaped path around the compressed breast and irradiating the compressed breast with an X-ray dose from the X-ray source located at a plurality of steps along the arc shaped path;
the step of detecting the X-rays transmitted through the breast comprises detecting the X-rays transmitted through the breast with a stationary digital X-ray detector;
the step of forming a first image of the X-ray modality comprises constructing a three dimensional image of the breast from a signal output by the digital X-ray detector;
the step of detecting gamma rays emitted from the compressed breast comprises detecting gamma rays which pass through a breast compression paddle with a nuclear medicine detector; and
the step of forming a second image of the nuclear medicine modality comprises constructing a three dimensional image of the breast from a signal output by the nuclear medicine detector.

38. The method of claim 37, further comprising:
moving the nuclear medicine detector away from the breast compression paddle prior to the step of irradiating the compressed breast with X-rays; and
moving the nuclear medicine detector into or over the breast compression paddle prior to the step of the step of detecting gamma rays.

39. The method of claim 33, further comprising:
fusing the first image and the second image to form a composite three dimensional image; and
displaying the fused image.

40. The method of claim 39, wherein fusion of the first and the second image is based on mechanically co-registered acquisition, co-registered acquisition supplemented by imaging physics or mutual information based registration.

41. The method of claim 33, further comprising using information from the first data set to acquire the second data set.

42. The method of claim 33, further comprising using information from the first data set to optimize quality of the second image.

43. The method of claim 33, wherein:
the first image comprises at least one of a CT image, an X-ray tomosynthesis image or a two dimensional X-ray mammography image; and
the second image comprises at least one of a SPECT image, a PET image, a positron emission mammography image, a scintimammography tomosynthesis image or a two dimensional scintimammography image.

44. The method of claim 33, further comprising acquiring at least one third data set of ultrasound modality.
